(19) United States
(12) Patent Application Publication
(10) Pub. No.: US 2025/0244817 A1
(43) Pub. Date: Jul. 31, 2025

(54) OPERATION MODE CONTROL METHOD FOR WEARABLE DEVICE, WEARABLE DEVICE AND MEDIUM

(71) Applicant: GOERTEK INC., Weifang, Shandong (CN)

(72) Inventor: Chenhong LIANG, Weifang, Shandong (CN)

(21) Appl. No.: 18/701,666

(22) PCT Filed: Dec. 16, 2021
(86) PCT No.: PCT/CN2021/138618

§ 371 Date: Apr. 16, 2024

(30) Foreign Application Priority Data
Oct. 31, 2021 (CN) 202111278699.4

(51) Int. Cl. G06F 1/3206; A61B 5/024; G06F 1/16

(52) U.S. Cl. CPC G06F 1/3206; A61B 5/024; G06F 1/163

(57) ABSTRACT

An operation mode control method for a wearable device, a wearable device and a medium, being applied to the technical field of wearable devices. The method comprises acquiring a physiological sign signal collected by a sensor in the current wearable device, wherein the physiological sign signal comprises at least a temperature signal (S11); determining whether the physiological sign signal satisfies a first preset condition, wherein the first preset condition comprises at least that an absolute value of a temperature difference corresponding to two adjacent temperature signals is greater than a first threshold (S12); if so, then controlling the operation mode of the current wearable device to be a low power consumption mode (S13). This control method determines the operation mode for controlling the current wearable device by means of changing the software, and adopts the temperature difference determination method to determine.

The present application claims the priority to the Chinese Patent Application No. 202111278699.4, entitled “operation mode control method for a wearable device, wearable device and medium”, submitted to the China Patent Office on Oct. 31, 2021, all contents of which are incorporated herein by reference.

### TECHNICAL FIELD

The present application relates to the technical field of wearable devices, and particularly, to an operation mode control method for a wearable device, a wearable device and a medium.

### BACKGROUND

In today's smart wearable devices such as watches, as their device functions increase. Various sensors configured to detect various data are also continuously used in wearable devices, which significantly reduces the standby time of wearable devices. When the sensors are operating, the self-awakening of the sensor may also lead to a reduction in the battery life of the wearable device, thus affecting the user experience.

In order to solve the problem of battery life of wearable devices, it is usually improved by means of hardware. For example, low-power components are chosen to replace the original high-power components to operate, and thereby achieve the purpose of reducing power consumption. However, on the one hand, this method needs to replace the hardware, and on the other hand, the requirements for hardware will also increase accordingly, thereby increasing research and development costs.

Therefore, how to improve the battery life is an urgent technical problem needed to be solved by those skilled in the art.

### SUMMARY

A purpose of the present application is to provide an operation mode control method for a wearable device, a wearable device and a medium to improve the battery life of the wearable device.

In order to solve the above technical problem, the present application provides an operation mode method for wearable device, comprising:
acquiring physiological sign signals collected by a sensor in the current wearable device, wherein the physiological sign signal comprises at least a temperature signal;
determining whether the physiological sign signal satisfies a first preset condition, wherein the first preset condition comprises at least that an absolute value of a temperature difference corresponding to two adjacent temperature signals is greater than a first threshold; if so, then controlling the operation mode of the current wearable device to be a low power consumption mode, wherein the operation mode comprises a normal operation mode and the low power consumption mode, and the number of components in an operating state in the low power consumption mode is less than the number of components in an operating state in the normal operation mode.

Preferably, the physiological sign signal further comprises a heart rate signal, and the first preset condition further comprises a case that the absolute value of the temperature difference corresponding to the two adjacent temperature signals is greater than the first threshold and a pulse signal generated within a first preset time by the heart rate signal is interrupted.

Preferably, in a case that the absolute value of the temperature difference corresponding to the two adjacent temperature signals is greater than the first threshold, then acquiring the heart rate signal to determine whether the pulse signal generated within the first preset time by the heart rate signal is interrupted.

Preferably, the method further comprises:
when the physiological sign signal satisfies a second preset condition, controlling the operation mode of the current wearable device to be the normal operation mode, wherein the second preset condition comprises at least that the absolute value of the temperature difference corresponding to the two adjacent temperature signals is greater than a second threshold.

Preferably, the physiological sign signal further comprises the heart rate signal, and the second preset condition further comprises a case that the absolute value of the temperature difference corresponding e two adjacent temperature signals is greater than the second threshold and the pulse signal generated within a second preset time by the heart rate signal is not interrupted.

Preferably, in a case that the absolute value of the temperature difference corresponding to the two adjacent temperature signals is greater than the second threshold, then acquiring the heart rate signal to determine whether the pulse signal not generated within the second preset time by the heart rate signal is interrupted.

In order to solve the above technical problems, the present application also provides a wearable device, comprising:
an acquisition module configured to acquire a physiological sign signal collected by a sensor in the current wearable device, wherein the physiological sign signal comprises at least a temperature signal;
a determination module configured to determine whether the physiological sign signal satisfies a first preset condition, wherein the first preset condition comprises at least that an absolute value of a temperature difference corresponding to the two adjacent temperature signals is greater than a first threshold, and if so, then a control module is activated; and
the control module configured to control an operation mode of the current wearable device to be a low power consumption mode, wherein the operation mode comprises a normal operation mode and the low power consumption mode, and the number of components in an operating state in the low power consumption mode is less than the number of components in an operating state in the normal operating mode.

In order to solve the above technical problems, the present application further provides a wearable device, comprising:
a memory configured to store a computer program;
a processor configured to implement steps of the operation mode control method for the wearable device as described above when executing the computer program.

In order to solve the above technical problems, the present application also provides a computer-readable storage medium, wherein a computer program is stored in the computer-readable storage medium, and when the computer program is executed by a processor, the steps of the operation mode control method for the wearable device as described above are implemented.

The operation mode control method for a wearable device according to the present application comprises: acquiring physiological sign signals collected by a sensor in the current wearable device, wherein the physiological sign signal comprises at least a temperature signal; determining whether the physiological sign signal satisfies a first preset condition, wherein the first preset condition comprises at least that an absolute value of a temperature difference corresponding to two adjacent temperature signals is greater than a first threshold; if so, then controlling the operation mode of the current wearable device to be a low power consumption mode, wherein the operation mode comprises a normal operation mode and the low power consumption mode, and the number of components in an operating state in the low power consumption mode is less than the number of components in an operating state in the normal operation mode. Therefore, this method determines the operation mode for controlling the current wearable device by means of changing the software, and adopts the temperature difference determination method to determine, thereby it can avoid the problem of erroneous determination caused by using temperature thresholds. In summary, the use of this technical solution not only increases the battery life of the wearable device, but also avoids changing the hardware and reduces the risk of erroneous determination, thereby improving the user experience.

In addition, the present application also provides a wearable device and a medium, which have the same beneficial effects as operation mode control method for the wearable device described above.

### BRIEF DESCRIPTION OF DRAWINGS

In order to explain the embodiments of the present application or the technical solutions in the prior art more clearly, the drawings needed to be used in the description of the embodiments or the prior art will be briefly introduced below. Obviously, the drawings in the following description are only part of the drawings of the present application. For those of ordinary skill in the art, other drawings can be obtained based on the provided drawings without exerting creative efforts.

FIG. 1 is a flow chart of an operation mode control method for a wearable device according to an embodiment of the present application;

FIG. 2 is a structural diagram of a wearable device according to an embodiment of the present application;

FIG. 3 is a structural diagram of another wearable device according to an embodiment of the present application;

FIG. 4 is a flow chart of another operation mode control method for a wearable device according to an embodiment of the present application.

### DETAILED DESCRIPTIONS

The technical solutions in the embodiments of the present application will be described below with reference to the accompanying drawings in the embodiments of the present application. Obviously, the described embodiments are only some of the embodiments of the present application, rather than all the embodiments. Based on the embodiments in the present application, all other embodiments acquired by those of ordinary skill in the art without creative efforts fall within the protection scope of the present application.

The core of the present application is to provide an operation mode control method for a wearable device, a wearable device and a medium to improve the battery life of the wearable device.

In order to enable those skilled in the art to better understand the solution of the present application, the present application will be further described in detail below in conjunction with the accompanying drawings and embodiments.

It should be noted that the wearable device according to the present application may be headphones, true wireless stereo (True Wireless Stereo, TWS) headsets, smart bracelets, smart glasses, smart sports watches, etc. The operation mode control method for the wearable device according to the present application is to control the operation mode of the wearable device by means of software to reduce power consumption to achieve the purpose of improving battery life.

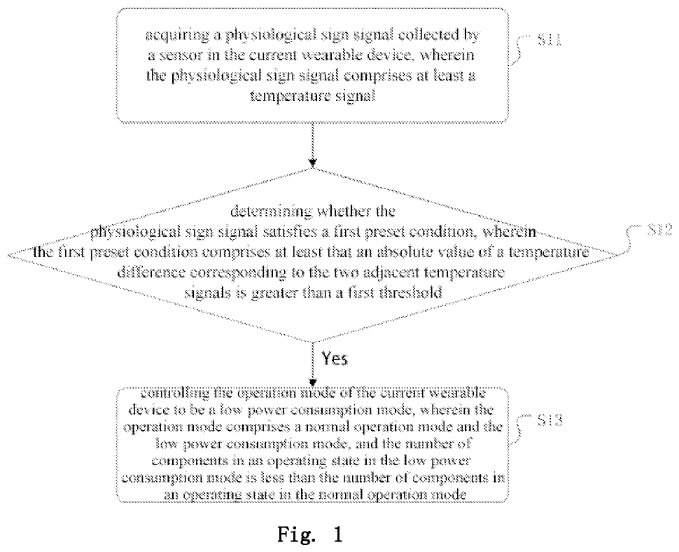

FIG. 1 is a flow chart of an operation mode control method for a wearable device according to an embodiment of the present application. As shown in FIG. 1, the present application provides an operation mode control method for a wearable device, which comprises:
S11: acquiring physiological sign signals collected by the sensor in the current wearable device. The physiological sign signal at least comprises temperature signal.

It can be understood that the present application controls the operation mode of the wearable device by means of software, and takes the signals collected by the sensor as basis. The wearable device comprises a signal acquisition module, a signal processing module and a signal recognition module, and the signal acquisition module collects signals by the sensor. The collected signals are transmitted to the signal processing module, the collected signals are converted into electrical signals, and the signal recognition module recognizes that the converted electrical signals are converted into digital signals of corresponding control operation modes.

The physiological sign signal collected by the sensor in the current wearable device is a signal unique to living organisms, comprising four major physiological signs, i.e., body temperature, pulse, blood pressure and respiration. It should be noted that heart rate is the frequency of heart beating. Pulse is the frequency of blood vessel beating. Under normal circumstances, pulse and heart rate are consistent. The blood pumped out each time the heart beats hits the blood vessel wall, the resulting change in pressure is the pulse that can be touched on the body surface. For example, the wearable device is a smart sports watch, which is worn on the user's wrist. The sensor of the wearable device collects the pulse signal to use as a normal heart rate signal, therefore the present application only considers normal situations.

It should be noted that the physiological sign signal at least comprises a temperature signal, which can be one or more different types of signals. For example, it only comprises a temperature signal or comprises a temperature signal in combination with a heart rate signal. The sensor that collects the temperature signal may be a body temperature negative temperature system sensor (Negative Temperature Coefficient, NTC). As the temperature increases, the resistance value decreases. The resistance value may change as the temperature changes. The electrical signal is converted into a digital signal by an analog to digital converter (Analog to Digital, A/D), and then the relevant algorithm of the software is used to acquire the current temperature value by self-calibration and self-compensation to determine the user's body temperature status. See the description below for specific implementation details. In addition, sensors that collect the physiological sign signals are different from sensors that collect other signals. The sensors that collect physiological sign signals can be temperature sensor, heart rate sensor, pressure sensor, respiration sensor, etc. Sensor that collects other signals is, for example, infrared sensor. The infrared sensor only uses infrared rays for data processing and non-contact temperature measurement. In addition to living organisms, other inanimate objects can be analyzed, such as gas composition analysis, etc. Therefore, physiological sign signals are not suitable to be collected by the infrared sensor.

For the collection of physiological sign signals, since the original physiological sign signal collected by the sensor may have signals such as burrs, noise, etc., filtering and preprocessing are performed in the signal processing module to obtain the physiological sign signal. Physiological sign signals comprise different types of signals, therefore the processed physiological sign signals are stored in the storage unit of the wearable device to facilitate calling.

The processed physiological sign signal is acquired, and different types of physiological sign signals are called in the storage unit of the wearable device for recognition processing. If the physiological sign signal calls only one type of temperature signal, it will be directly recognized to control the operation mode of the wearable device. If it calls two different types of signals, such as temperature and respiration signals, it can be acquired by setting priorities or performing recognition processing at the same time. After satisfying one or two conditions, the operation mode of the wearable device can be controlled, and it can set according to the actual situation. The present application does not have specific limitations. It should be noted that the physiological sign signals are collected and processed at the same time and then are put into the database and called according to the actual situation. The present application only acquires the physiological sign signals collected by the sensors in the current wearable device.

S12: determining whether the physiological sign signal satisfies a first preset condition, wherein the first preset condition comprises at least that the absolute value of the temperature difference corresponding to two adjacent temperature signals is greater than the first threshold; if so, then proceeding to step S13.

It is determined whether the acquired physiological sign signal satisfies the first preset condition according to the acquired physiological sign signal. The first preset condition is set for the physiological sign signal, and may be the basic value of the temperature signal, and the difference between the temperature signals before and after. The basic value of the respiratory frequency may also be the case where the pulse signal of the heart rate signal is not continuous, that is, being interrupted, etc. For example, the basic value of the temperature signal is set as follows: the normal value of temperature applicable to human body is 36° C. to 37.2° C., and the basic value is set to 36° C. When the user is in the external environment in winter, the body temperature often is lower than 36° C., which is easy to satisfy the preset conditions, and frequently controls the operation mode of the wearable device. Since the human body temperature will be maintained at a constant temperature for a period of time, therefore, if the current wearable device is in the normal operation mode, once the body temperature is lower than 36° C., the current wearable device may be mistakenly operated to enter a low power consumption mode. Erroneous operation may cause increased power consumption and even affect the user experience.

Physiological sign signal at least comprises a temperature signal, and the first preset condition at least comprises that the absolute value of the temperature difference corresponding to two adjacent temperature signals is greater than the first threshold.

Two adjacent temperature signals are acquired from the collected temperature signals. The temperature signals are collected according to a certain time. For example, a temperature signal is collected once according to 1 s. The two adjacent temperature signals are the temperature value (36° C. and 32° C.) corresponding to the temperature signal of 1 s before and after, that is, 1 s before and 1 s after. The temperature difference therebetween is the temperature difference 4° C. obtained by subtracting the temperature value 32° C. corresponding to the 1 s after from the temperature value 36° C. corresponding to the Is before, and the first threshold is set to 3° C. The absolute value of the temperature difference corresponding to the two adjacent temperature signals is 4° C., which is greater than the first threshold. If the temperature difference between the temperature signals 1 s after and 1 s before is −4° C., the temperature difference in the form of an absolute value is 4° C., which is greater than the first threshold, therefore, the temperature signal satisfies the first preset condition. Then, the operation mode of the current wearable device is controlled to be a low power consumption mode. If the first preset condition is not met, the current operating status is maintained.

It should be noted that the temperature difference corresponding to two adjacent temperature signals is based on the time sequence. The temperature signal collected later is subtracted from the temperature signal collected in the prior to obtain the temperature difference, and then the absolute value of the temperature difference is acquired and compared with the first threshold. The collected temperature signals are acquired according to a set interval time, and the first threshold is set according to the actual situation. The present application does not have specific limitations.

In addition, the physiological sign signal may also be a temperature signal and a heart rate signal, and the first preset condition further comprises a case that the absolute value of the temperature difference corresponding to the two adjacent temperature signals is greater than the first threshold and the pulse signal generated within the first preset time by the heart rate signal is interrupted.

Therefore, the first preset condition can be set according to the actual situation, comprising at least one type of signal, that is, temperature signal, and can also comprise a combination of two different types of signals, that is, temperature signal and heart rate signal. The preset condition may be set for only the temperature signal or for combination of the temperature signal and the heart rate signal, without specific limitations.

S13: controlling the operation mode of the current wearable device to a low power consumption mode, wherein the operation mode comprises a normal operation mode and the low power consumption mode, and the number of components in the operating state in the low power consumption mode is less than the number of components in an operating state in the normal operation mode.

The operation mode of the current wearable device comprises the normal operation mode and the low power consumption mode. The normal operation mode means that all the components in the current wearable device operate normally. The low power consumption mode means that the number of components in the operating state in the current wearable device is less than the number of components in an operating state in the normal operation mode. It can be understood that different wearable devices have different components included therein, but they all need to comprise sensors that collect physiological sign signals. In the low power consumption mode, the components in the operating state in the current wearable device may comprise sensors that collect physiological sign signals. For example, the physiological sign signals comprise temperature signal and heart rate signal. In the low power consumption mode, both the temperature sensor and the heart rate sensor are in the operating state. The wearable device may also comprise components other than the sensors that collect physiological sign signals. It should be noted that, the foregoing content is only a specific embodiment. In other embodiments, in the low power consumption mode, all the components (comprising the sensors that collect physiological sign signals) can also be turned off, that is, shut down. Considering the usage convenience of users, generally, at least the sensor that collect the physiological sign signal and microcontroller unit (MCU) should be retained so that when the collected physiological sign signals satisfy the second preset condition described below, the current wearable device will automatically be switched from the current low power consumption mode to the normal power consumption mode, as described below for details. In addition, in the low power consumption mode, there is no limitation to the components that are turned off. For example, for a bracelet, the components that can be turned off are acceleration sensor, voice collection module, etc.

When the physiological sign signal satisfies the first preset condition, the operation mode of the current wearable device is controlled to be the low power consumption mode, which is divided into two types, one type is to adjust the normal operation mode to the low power consumption mode, and the other type is to continue to maintain the low power consumption mode if it has been in the low power consumption mode.

In addition, when the physiological sign signal does not satisfy the first preset condition, the current wearable device can maintain the current operation mode or control the operation mode of the current wearable device to the normal operation mode. Therefore, controlling the operation mode of the current wearable device is implemented according to the specific content of the first preset condition. The different content of different types of signals in the first preset condition results in different operation modes. It can be set according to the specific actual situation. The present application does not have specific limitations.

When the operation mode of the current wearable device is the low power consumption mode, if the wearable device is switched from the low power consumption mode to the normal operation mode, the switch can be automatically adjusted according to the fact that the physiological sign signal satisfies another preset condition, or the operation mode of the current wearable device is adjusted manually through a button disposed in the wearable device according to the user's needs. The present application does not have specific limitations.

The operation mode control method for the wearable device according to the present application comprises acquiring a physiological sign signal collected by a sensor in the current wearable device, wherein the physiological sign signal comprises at least a temperature signal; determining whether the physiological sign signal satisfies a first preset condition, wherein the first preset condition comprises at least that an absolute value of a temperature difference corresponding to two adjacent temperature signals is greater than a first threshold; if so, then controlling the operation mode of the current wearable device to be a low power consumption mode, wherein the operation mode comprises a normal operation mode and the low power consumption mode, and the number of components in an operating state in the low power consumption mode is less than the number of components in an operating state in the normal operation mode. Therefore, this method determines the operation mode for controlling the current wearable device by means of changing the software, and adopts the temperature difference determination method to determine, thereby it can avoid the problem of erroneous determination caused by using temperature thresholds. In summary, the use of this technical solution not only increases the battery life of the wearable device, but also avoids changing the hardware and reduces the risk of erroneous determination, improving the user experience. Based on the above embodiments, the physiological sign a signal comprises temperature signal as well as a heart rate signal. The sensor that collects the heart rate signal is a photoelectric sensor. After irradiating a beam of a certain wavelength to the skin surface, the photoelectric sensor receives the intensity of the returned light to detect the heart rate signal. During this process, the light will be attenuated through absorption by the skin, muscles and blood. As the heart contracts, the heart rate signal may show a continuous pulse signal. The physiological sign signal is temperature signal and heart rate signal, and the corresponding first preset condition is specifically:

The absolute value of the temperature difference corresponding to two adjacent temperature signals is greater than the first threshold and the pulse signal generated within the first preset time by the heart rate signal is interrupted.

The preset condition for the temperature signal has been described in detail in the above embodiments and will not be repeated here. The collection mode of the heart rate signal is the same as that of the temperature signal. The heart rate signal is collected according to the first timing, and it is detected whether the pulse signal generated within the first preset time by the collected heart rate signal is interrupted. If the pulse signal generated within the first preset time is an interrupted pulse signal, it means that the current wearable device is not worn on the user's wrist.

For example, the heart rate signal is acquired once every 1 minute according to the first timing, and the pulse signal generated by the heart rate signal is detected within the first preset time of 30 minutes. If there are continuous pulse signals in the ahead 5 minutes of the 30 minutes and a continuous pulse signal appears from the 7th minute to the 30th minute but an interruption occurs between the 5th minute and the 7th minute, it is the case that the pulse signal generated within the first preset time is interrupted. The collection of the heart rate signal at the first timing and the specific value of the first preset time are set according to the actual situation, and there are no specific limitations.

It should be noted that the first preset condition for the heart rate signal is set as that the temperature difference corresponding to two adjacent temperature signals is greater than the first threshold. If the temperature signal satisfies the condition that the temperature difference corresponding to two adjacent temperature signals is greater than the first threshold, then the heart rate signal according to the first timing is extracted and the condition that the pulse signal generated within the first preset time is interrupted is detected. If the temperature signal and the heart rate signal satisfy the first preset condition, then the operation mode of the current wearable device is controlled to be the low power consumption mode. When only one of the temperature signal and the heart rate signal satisfies the preset condition corresponding to the first preset condition, the current wearable device continues to maintain the original operation mode without any change.

In a specific embodiment, the sensor that collects physiological sign signals is always in collection operation. The physiological sign signal is extracted in the signal processing module and the signal recognition module according to the actual situation. The physiological sign signal is extracted according to different timing intervals, and the present application does not have specific limitations.

The first condition according to the present application is that the absolute value of the temperature difference corresponding to two adjacent temperature signals is greater than the first threshold and the pulse signal generated within the first preset time by the heart rate signal is interrupted. When the temperature signal and the heart rate signal satisfy the first preset condition, the operation mode of the current wearable device is controlled to be the low power consumption mode, the operation mode of the current wearable device is further r accurately control, to reduce power consumption, so as to achieve the effect of increasing the battery life of the wearable device and improving user experience.

Based on the above embodiments, the physiological sign signal comprises temperature signal and heart rate signal. Specific embodiment thereof is as follows:

The absolute value of the temperature difference corresponding to two adjacent temperature signals is greater than the first threshold, then acquiring the heart rate signal to determine whether the pulse signal generated within the first preset time by the heart rate signal is interrupted.

In a case that the absolute value of the acquired temperature difference corresponding to two adjacent temperature signals is greater than the first threshold, the heart rate signal is further acquired, and it is determined whether the pulse signal generated within the first preset time by the heart rate signal is interrupted. It should be noted that when the temperature signal satisfies the condition that the absolute value of the temperature difference is greater than the first threshold, the heart rate sensor is turned on to acquire the heart rate signal, and it is determined whether the pulse signal generated within the first preset time by the heart rate signal is interrupted. At the time of acquiring the temperature signal, only the temperature signal is acquired. When the temperature signal satisfies a certain condition, then the heart rate signal is acquired.

In addition, the detailed embodiment of the acquisition of the heart rate signal, and the content that the absolute value of the temperature difference corresponding to two adjacent temperature signals is greater than the first threshold, and the pulse signal generated within the first preset time by the heart rate signal is interrupted, has been described as above and will not be repeated here.

When both the temperature signal and the heart rate signal satisfy the preset condition, the operation mode of the current wearable device is controlled to be the low power consumption mode, that is, the remaining sensors except the sensor that collects physiological sign signal are turned off. When the operation mode of the current wearable device is the low power consumption mode, it is necessary to turn off the remaining sensors except the sensor that collects the physiological sign signal. If the physiological sign signal is a temperature signal, the remaining sensors except the body temperature NTC need to be turned off. If the physiological sign signal is temperature signal and heart rate signal, the remaining sensors except the body temperature NTC and heart rate sensor are turned off. In the low power consumption mode, the sensor that collects physiological sign signal is retained and the remaining sensors are turned off.

Power management integrated circuit (power management integrated circuit, PMIC) serves as the power manager to turn off the remaining sensors. The PMIC supplies power to the sensors of the current wearable device. The PMIC has the characteristics of small size and high application efficiency. The use of PMIC as the power manager in the present application is merely a preferred embodiment.

The controller of the current wearable device transmits an operate instruction to the power manager PMIC through the serial port bus inter-integrated circuit (Inter-Integrated Circuit, I2C) protocol to turn off the remaining sensors except for the sensor that collects physiological sign signal. In the present application, use of the I2C protocol as the serial port bus communication is merely a preferred embodiment.

According to the present application, when the absolute value of the temperature difference corresponding to two adjacent temperature signals is greater than the first threshold, then acquiring the heart rate signal to determine whether the pulse signal generated within the first preset time by the heart rate signal is interrupted. When the temperature signal satisfies the condition, the heart rate sensor is turned on, and then it is determined whether the pulse signal generated within the first preset time by the heart rate signal is interrupted. If so, the operation mode of the current wearable device is controlled to be the low power consumption mode. When the temperature signal satisfies the condition, the heart rate sensor is turned on and it is determined whether the heart rate signal satisfies the condition, the operation mode of the current wearable device is further accurately controlled, to reduce power consumption and improve the battery life of the wearable device.

After the operation mode of the wearable device is the low power consumption mode, the method further comprises:

When the physiological sign signal satisfies the second preset condition, the operation mode of the current wearable device is controlled to be the normal operation mode, wherein the second preset condition comprises at least that the absolute value of the temperature difference corresponding to two adjacent temperature signals is greater than the second threshold.

The physiological sign signal at least comprises a temperature signal. The collection of the temperature signal is described in detail in the above embodiments and will not be repeated. The second preset condition at least comprises that the absolute value of the temperature difference corresponding to two adjacent temperature signals is greater than the second threshold. Two adjacent temperature signals are acquired from the collected temperature signals. The temperature signals are collected according to a certain time. For example, a temperature signal is collected once according to 1 s. The two adjacent temperature signals are the temperature values corresponding to the temperature signal of 1 s before and after, that is, 1 s before and 1 s after, which are 32° C. and 36° C., respectively. The temperature difference therebetween is the temperature difference −4° C. acquired by subtracting the temperature value 36° C. corresponding to the 1 s after from the temperature value 32° C. corresponding to the 1 s before, the absolute value thereof is 4° C. and the second threshold is set to 3° C. Therefore, the absolute value 4° C. of the temperature difference corresponding to the two adjacent temperature signals is greater than the second threshold 3° C.

It should be noted that the temperature difference corresponding to two adjacent temperature signals is based on the time sequence. The temperature signal collected later is subtracted from the temperature signal collected in the prior to obtain the temperature difference, and then the absolute value of the temperature difference is acquired and compared with the second threshold. The collected temperature signals are acquired according to a set interval time, and the second threshold may be equal to the first threshold or may be set according to the actual situation.

The operation mode of the current wearable device is the low power consumption mode. At this time, the sensor in the operating state in the current wearable device is the sensor that collects physiological sign signal. Therefore, when the physiological sign signal satisfies the second preset condition, the operation mode of the current wearable device is controlled to be the normal operation mode. The normal operation mode is a mode in which the sensor that collects physiological sign signal and the remaining sensors in the current wearable device are all in normal operating status.

According to the fact that the acquired physiological sign signal satisfies the second preset condition, the second preset condition is set for the physiological sign signal, which can be a temperature signal, or two types of signals: temperature signal and heart rate signal, or a combination of more than two types of signals. There are no specific limitations thereto.

It should be noted that, based on the above embodiments, when the physiological sign signal does not satisfy the first preset condition, it does not mean that the second preset condition can be met, and it needs to be set according to the specific actual situation.

When the operation mode of the current wearable device is the normal operation mode, the sensor that collects physiological sign signal and the remaining sensors are turned on to operate, and the power manager thereof supplies power to the sensors of the current wearable device. It can be understood that when the low power consumption mode is switched to the normal operation mode or the normal operation mode is switched to the low power consumption mode, prompt information can be set. The types of the prompt information can be broadcasting voice information to inform the user, providing a flashing device on the display screen of the wearable device, or activating a vibration module to remind the user, thereby improving the user experience.

According to the present application, when the physiological sign signal satisfies the second preset condition, the operation mode of the current wearable device is controlled to be the normal operation mode, and the operation mode of the current wearable device is controlled by means of software to improve the user experience.

Based on the above embodiments, the acquired physiological sign signal comprises temperature signal and heart rate signal. The collection of the temperature signal and heart rate signal has been described in detail in the above embodiments and will not be repeated here. The second preset condition is as follows.

The absolute value of the temperature difference corresponding to two adjacent temperature signals is greater than the second threshold and the pulse signal generated within the second preset time by the heart rate signal is not interrupted.

The absolute value of the temperature difference corresponding to two adjacent temperature signals being greater than the second threshold has been described in detail in the above embodiments and will not be repeated here. The present application does not have specific limitations on acquiring the temperature signal and the heart rate signal at set intervals, and on the first threshold and the second threshold.

On this basis, the heart rate signal is collected according to the second timing, and it is detected that the pulse signal generated within the second preset time by the collected heart rate signal is not interrupted, that is, the heart rate signal is a continuous pulse signal, for example, the second timing is 5 s acquiring once, and the pulse signal generated by the heart rate signal is detected within the second preset time of 2 minutes. If there is no continuous pulse signal having interruption within 2 minutes, that is, it is a continuous pulse signal from the 0th minute to the 2nd minute, there is no interruption, it means that the current wearable device is worn on the user's wrist.

It can be understood that the heart rate signal within the first preset condition is collected according to the first timing, and the heart rate signal within the second preset condition is collected according to the second timing, wherein the first timing is greater than the second timing. In order to control the operation mode of the current wearable device more accurately, the first preset condition is that controlling the operation mode of the current wearable device to be the low power consumption mode. The first timing is set longer to ensure accurate control of the low power consumption mode. The second preset condition is that controlling the operation mode of the current wearable device to be the normal operation mode. The second timing is set shorter, the operation mode is control immediately to be switched to the normal operation mode once the heart rate signal appears a continuous pulse signal to ensure the user experience, the first timing and the second timing are not specified in the present application and are set according to the actual situation.

In addition, the physiological sign signal in this embodiment is a temperature signal and a heart rate signal. Correspondingly, when the physiological sign signal in the above embodiment is a temperature signal, the second preset condition is that the absolute value of the temperature difference corresponding to two adjacent temperature signals is greater than the second threshold, which will not be repeated here.

The second preset condition according to the present application is that the absolute value of the temperature difference corresponding to two adjacent temperature signals is greater than the second threshold and the pulse signal generated within the second preset time by the heart rate signal is not interrupted. When the temperature signal and the heart rate signal satisfy the second preset condition, the operation mode of the current wearable device is controlled to the normal operation mode, the operation mode of the current wearable device is further accurately controlled, thereby improving the user experience.

In the above embodiment, the physiological sign signal comprises the temperature signal and the heart rate signal. Specific embodiment thereof is as follows:

In the case where the absolute value of the temperature difference corresponding to two adjacent temperature signals is greater than the second threshold, then acquiring the heart rate signal to determine whether the pulse signal generated within the second preset time by the heart rate signal is not interrupted.

In the case where the absolute value of the temperature difference corresponding to two adjacent temperature signals is greater than the second threshold, the heart rate signal is further acquired, and it is determined whether the pulse signal generated within the second preset time by the heart rate signal is not interrupted. It should be noted that, when the temperature signal satisfies the condition that the absolute value of the temperature difference is greater than the second threshold, the heart rate sensor is turned on to acquire the heart rate signal, and it is determined whether the pulse signal generated within the second preset time by the heart rate signal is not interrupted. When acquiring the temperature signal, only the temperature signal is acquired. When the temperature signal satisfies a certain condition, then the heart rate signal is acquired.

In addition, the detailed embodiment of the acquisition of the heart rate signal, and the content that the absolute value of the temperature difference corresponding to the two adjacent temperature signals is greater than the second threshold, and the pulse signal generated within the second preset time by the heart rate signal is not interrupted has been described in the above and will not be repeated here.

According to the present application, in the case where the absolute value of the temperature difference corresponding to two adjacent temperature signals is greater than the second threshold, then acquiring the heart rate signal to determine whether the pulse signal generated within the second preset time by the heart rate signal is not interrupted. When the temperature signal satisfies the condition, then the heart rate sensor is turned on, and it is determined whether the pulse signal generated within the second preset time by the heart rate signal is not interrupted. If so, the operation mode of the current wearable device is controlled to be the normal operation mode. When the temperature signal satisfies the condition, then the heart rate sensor is turned on and it is determined whether the heart rate signal satisfies the condition, the operation mode of the current wearable device is further accurately controlled, thereby reducing power consumption and improving the battery life of the wearable device.

Based on the above embodiments, the physiological sign signal is the temperature signal and the heart rate signal, which control the settings of the first preset time in the first preset condition when the operation mode of the current wearable device is the low power consumption mode and the second preset time in the second preset condition when the operation mode of the current wearable device is the normal operation mode. The first preset time is greater than the second preset time.

When the temperature signal satisfies the first preset condition, the heart rate signal is extracted and the condition that the pulse signal generated within the first preset time is interrupted is detected. Here, the first preset time is set longer in order to acquire the result of the current wearing status of the wearable device more accurately, so as to control the operation mode of the current wearable device according to the wearing status.

When the temperature signal satisfies the second preset condition, the heart rate signal is extracted and the condition that the pulse signal generated within the second preset time is not interrupted is detected, wherein the second preset time is set shorter than the first preset time to prevent the user experience from being deteriorated due to longer detection time caused by longer second preset time set in the wearable device when the user has worn the current wearable device. It should be noted that this embodiment is not applicable when the physiological sign signal is only a temperature signal. The first preset time is greater than the second preset time. The specific values of the first preset time and the second preset time are not specified in the present application and are set according to the actual situation.

According to the present application, the first preset time is greater than the second preset time, so as to acquire the wearing status of the current wearable device more accurately in a timely manner to control the operation mode of the current wearable device more efficiently and improve the user experience.

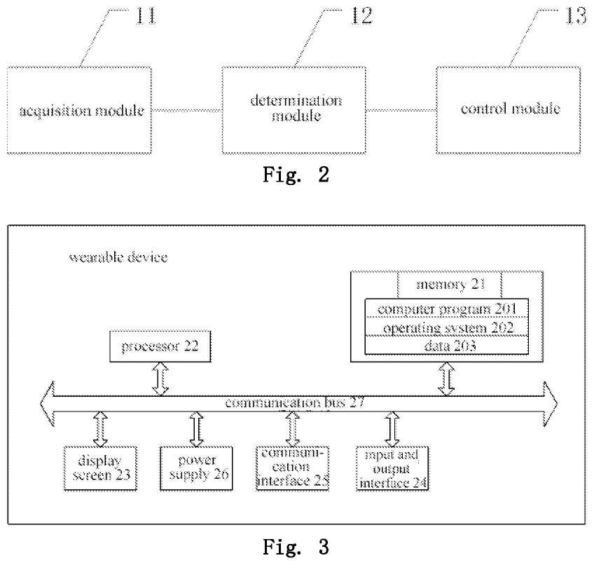

Various embodiments corresponding to the operation mode control method for the wearable device are described in detail above. On this basis, the present application also discloses a wearable device corresponding to the above method. FIG. 2 is a structural diagram of the wearable device according to an embodiment of the present application. As shown in FIG. 2, the wearable device comprises:
an acquisition module 11 configured to acquire a physiological sign signal collected by a sensor in the current wearable device, wherein the physiological sign signal comprises at least a temperature signal;
a determination module 12 configured to determine whether the physiological sign signal satisfies a first preset condition, wherein the first preset condition comprises at least that an absolute value of a temperature difference corresponding to two adjacent temperature signals is greater than a first threshold, and if so, then a control module 13 is activated;
the control module 13 configured to control the operation mode of the current wearable device to be a low power consumption mode, wherein the operation mode comprises a normal operation mode and the low power consumption mode, and the number of components in an operating state in the low power consumption mode is less than the number of components in an operating state in the normal operating mode.

The embodiments of the device correspond to the above-mentioned embodiments, therefore, for the embodiments of the device, please refer to the descriptions of the embodiments of the above-mentioned method, and will not be repeated here.

The present application provides a wearable device that acquires a physiological sign signal collected by a sensor in the current wearable device, wherein the physiological sign signal comprises at least a temperature signal; determines whether the physiological sign signal satisfies a first preset condition, wherein the first preset condition comprises at least that an absolute value of a temperature difference corresponding to two adjacent temperature signals is greater than a first threshold; if so, then controls the operation mode of the current wearable device to be a low power consumption mode, wherein the operation mode comprises a normal operation mode and the low power consumption mode, and the number of components in an operating state in the low power consumption mode is less than the number of components in an operating state in the normal operation mode. Therefore, this wearable device determines the operation mode for controlling the current wearable device by means of changing the software, and adopts the temperature difference determination method to determine, thereby it can avoid the problem of erroneous determination caused by using temperature thresholds. In summary, the use of this technical solution not only increases the battery life of the wearable device, but also avoids changing the hardware and reduces the risk of erroneous determination, improving the user experience.

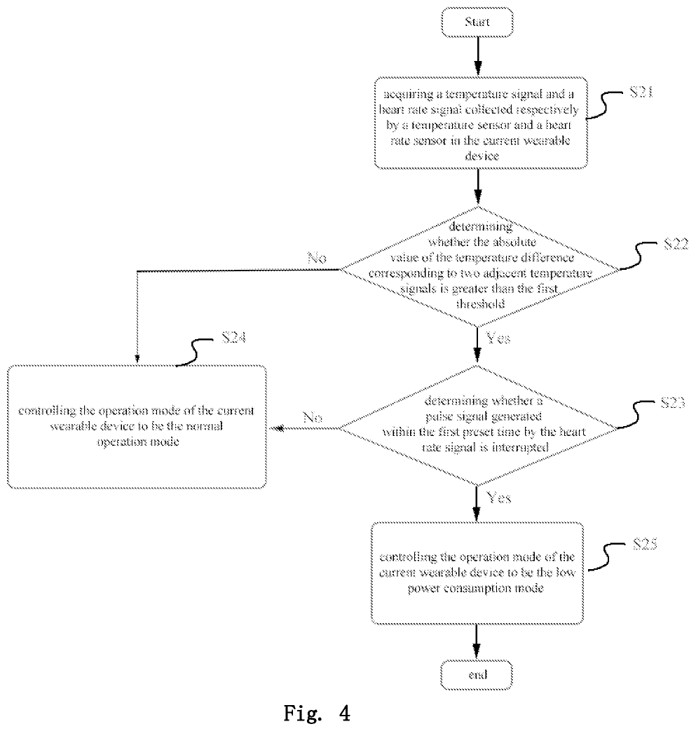

Please refer to FIG. 3, which is a structural diagram of another wearable device according to an embodiment of the present application. As shown in FIG. 3, the wearable device comprises:
a memory 21 configured to store a computer program;
a processor 22 configured to implement steps of the operation mode control method for the wearable device as described above when executing the computer program.

The wearable device according to the present embodiment may comprise but are not limited to headphones, TWS headphones, smart bracelets, smart sports watches, etc.

Here, the processor 22 may comprise one or more processing cores, such as a 4-core processor, an 8-core processor, etc. The processor 22 can be implemented by at least one hardware form among digital signal processing (DSP), field-programmable gate array (FPGA), and programmable logic array (PLA). accomplish. The processor 22 may also comprise a main processor and a co-processor. The main processor is a processor configured to process data in the wake-up state, also called a central processing unit (Central Processing Unit, CPU); the co-processor is a low-power processor configured to process data in the standby state. In some embodiments, the processor 22 may have a graphics processor (Graphics Processing Unit, GPU) integrated therein, and the GPU is responsible for rendering and drawing content to be displayed on the display screen. In some embodiments, the processor 22 may also comprise an artificial intelligence (Artificial Intelligence, AI) processor, which is configured to process computing operations related to machine learning.

Memory 21 may comprise one or more computer-readable storage medium, which may be non-transitory. The memory 21 may also comprise high-speed random access memory, and non-volatile memory, such as one or more magnetic disk storage devices and flash memory storage devices. In this embodiment, the memory 21 is at least configured to store the following computer program 201. After the computer program is loaded and executed by the processor 22, the relevant steps of the operation mode control method for the wearable device disclosed in any of the foregoing embodiments can be implemented. In addition, the resources stored in the memory 21 may also comprise the operating system 202, data 203, etc., and the storage method may be short-term storage or permanent storage. Here, the operating system 202 may comprise Windows, Unix, Linux, etc. The data 203 may comprise but is not limited to data related to the operation mode control method for the wearable device, and the like.

In some embodiments, the wearable device may also comprise a display screen 23, an input-output interface 24, a communication interface 25, a power supply 26 and a communication bus 27.

Those skilled in the art can understand that FIG. 3 is a structural diagram of another wearable device according to an embodiment of the present application. The structure shown in FIG. 3 does not constitute a limitation to the wearable device, and may comprise more or fewer components than that shown in the Figures.

The processor 22 implements the operation mode control method for the wearable device according to any of the above embodiments by calling instructions stored in the memory 21.

The present application provides a wearable device that acquires a physiological sign signal collected by a sensor in the current wearable device, wherein the physiological sign signal comprises at least a temperature signal; determines whether the physiological sign signal satisfies a first preset condition, wherein the first preset condition comprises at least that an absolute value of a temperature difference corresponding to two adjacent temperature signals is greater than a first threshold; if so, then controls the operation mode of the current wearable device to be a low power consumption mode, wherein the operation mode comprises a normal operation mode and the low power consumption mode, and the number of components in an operating state in the low power consumption mode is less than the number of components in an operating state in the normal operation mode. Therefore, this wearable device determines the operation mode for controlling the current wearable device by means of changing the software, and adopts the temperature difference determination method to determine, thereby it can avoid the problem of erroneous determination caused by using temperature thresholds. In summary, the use of this technical solution not only increases the battery life of the wearable device, but also avoids changing the hardware and reduces the risk of erroneous determination, thereby improving the user experience.

In conjunction with the above embodiments, FIG. 4 is a flow chart of another operation mode control method for a wearable device according to an embodiment of the present application. As shown in FIG. 4, when the wearable device is in the normal operation mode, the physiological sign signal thereof is a temperature signal and a heart rate signal. When the preset condition of the temperature signal is met, the heart rate signal is acquired, to continue to detect whether the preset condition of the heart rate signal is met, until the preset conditions of both types of signals are met, in this case, the operation mode of the wearable device can be controlled to be a low power consumption mode, the method specifically comprises:
S21: acquiring a temperature signal and a heart rate signal collected respectively by a temperature sensor and a heart rate sensor in the current wearable device;
S22: determining whether the absolute value of the temperature difference corresponding to two adjacent temperature signals is greater than the first threshold; if so, proceeding to step S23, and if not, proceeding to step S24;
S23: determining whether a pulse signal generated within the first preset time by the heart rate signal is interrupted; if so, proceeding to step S25, and if not, proceeding to step S24;
S24: controlling the operation mode of the current wearable device to be the normal operation mode;
S25: controlling the operation mode of the current wearable device to be the low power consumption mode.

It should be noted that, in this embodiment, the current operation mode of the wearable device is switched from the normal operation mode to the low power consumption mode when the preset condition of the temperature signal in step S22 is met and furthermore the preset condition of the heart rate signal in step S23 is met.

A flow chart of another operation mode control method for a wearable device according to an embodiment of the present application is described above, it has the same beneficial effects as the above-mentioned operation mode control method for a wearable device.

Furthermore, the present application also provides a computer-readable storage medium. The computer-readable storage medium stores a computer program therein. When the computer program is executed by the processor 22, the steps of the above-mentioned operation mode control method for the wearable device are implemented.

It can be understood that if the method in the above embodiments is implemented in the form of software functional units and sold or used as independent product, they can be stored in a computer-readable storage medium. Based on this understanding, the part of the technical solution of the present application which is essential or contributes to the existing technology, or all or part of the technical solution can be embodied in the form of a software product, and the computer software product is stored in a storage medium, and performs all or part of the steps of the methods described in various embodiments of the present application. The afore-mentioned storage medium comprises medium that can store program codes such as USB disk, mobile hard disk, read-only memory (ROM), random access memory (RAM), magnetic disk or optical disk.

The computer-readable storage medium according to the present application may be referred to the above method embodiments, which will not be described in detail here. It has the same beneficial effects as that of the operation mode control method for the wearable device described above.

The operation mode control method for a wearable device, the wearable device and the medium according to the present application are described in detail above. Each embodiment in the specification is described in a progressive manner. Each embodiment focuses on its differences from other embodiments. The same and similar parts between the various embodiments can be referred to each other. As for the device disclosed in the embodiments, since it corresponds to the method disclosed in the embodiment, the description is relatively simple. For relevant details, please refer to the description in the method portion. It should be noted that for those of ordinary skill in the art, several improvements and modifications can be made to the present application without departing from the principles of the present application, and these improvements and modifications also fall within the protection scope of the claims of the present application.

It should also be noted that in this specification, relational terms such as first and second are only configured to distinguish one entity or operation from another entity or operation, and do not necessarily require or imply that these entities or operations have such actual relationship or sequence between them. Furthermore, the terms “comprise”, “include”, or any other variations thereof are intended to cover a non-exclusive inclusion, such that a process, method, article, or components that comprises several elements comprises not only those elements, but also other elements not expressly listed, or elements inherent to the process, method, article or equipment. Without further limitation, an element defined by the statement “comprises a . . . ” does not exclude the presence of additional identical elements in a process, method, article, or device that comprises the stated element.

1. An operation mode control method for a wearable device, comprising:
acquiring a physiological sign signal collected by a sensor in the current wearable device, wherein the physiological sign signal comprises at least a temperature signal;
determining whether the physiological sign signal satisfies a first preset condition, wherein the first preset condition comprises at least that an absolute value of a temperature difference corresponding to two adjacent temperature signals is greater than a first threshold, if so, then controlling an operation mode of the current wearable device to be a low power consumption mode, wherein the operation mode comprises a normal operation mode and the low power consumption mode, and the number of components in an operating state in the low power consumption mode is less than the number of components in an operating state in the normal operation mode.

2. The operation mode control method for the wearable device according to claim 1, wherein the physiological sign signal further comprises a heart rate signal, and the first preset condition further comprises a case that the absolute value of the temperature difference corresponding to the two adjacent temperature signals is greater than the first threshold and a pulse signal generated within a first preset time by the heart rate signal is interrupted.

3. The operation mode control method for the wearable device according to claim 2, wherein in a case that the absolute value of the temperature difference corresponding to the two adjacent temperature signals is greater than the first threshold, then acquiring the heart rate signal to determine whether the pulse signal generated within the first preset time by the heart rate signal is interrupted.

4. The operation mode control method for the wearable device according to claim 1, further comprising:
when the physiological sign signal satisfies a second preset condition, controlling the operation mode of the current wearable device to be the normal operation mode, wherein the second preset condition comprises at least that the absolute value of the temperature difference corresponding to the two adjacent temperature signals is greater than a second threshold.

5. The operation mode control method for the wearable device according to claim 4, wherein the physiological sign signal further comprises the heart rate signal, and the second preset condition further comprises a case that the absolute value of the temperature difference corresponding to the two adjacent temperature signals is greater than the second threshold and the pulse signal generated within a second preset time by the heart rate signal is not interrupted.

6. The operation mode control method for the wearable device according to claim 5, wherein in a case that the absolute value of the temperature difference corresponding to the two adjacent temperature signals is greater than the second threshold, then acquiring the heart rate signal to determine whether the pulse signal generated within the second preset time by the heart rate signal is not interrupted.

7. The operation mode control method for the wearable device according to claim 5, wherein the first preset time is greater than the second preset time.

8. A wearable device, comprising:
an acquisition module configured to acquire a physiological sign signal collected by a sensor in the current wearable device, wherein the physiological sign signal comprises at least a temperature signal;
a determination module configured to determine whether the physiological sign signal satisfies a first preset condition, wherein the first preset condition comprises at least that an absolute value of a temperature difference corresponding to the two adjacent temperature signals is greater than a first threshold, and if so, then a control module is activated; and
the control module configured to control an operation mode of the current wearable device to be a low power consumption mode, wherein the operation mode comprises a normal operation mode and the low power consumption mode, and the number of components in an operating state in the low power consumption mode is less than the number of components in an operating state in the normal operating mode.

9. A wearable device, comprising:
a memory configured to store a computer program;
a processor configured to implement steps of the operation mode control method for the wearable device according to claim 1 when executing the computer program.

10. (canceled)
